(19) United States
(12) Patent Application Publication
(10) Pub. No.: US 2002/0111645 A1
(43) Pub. Date: Aug. 15, 2002

(54) Vacuum prep catheter

(75) Inventors: Yiqun Wang, Maple Grove, MN (US); Daniel James Horn, Shoreview, MN (US); Daniel K. Tomaschko, Savage, MN (US); Richard J. Olson, Blaine, MN (US)

Correspondence Address: David M. Crompton, CROMPTON, SEAGER & TUFTE, LLC, Suite 895, 331 Second Avenue South, Minneapolis, MN, 55401-2246, US

(73) Assignee: SciMed Life System, Inc.

(21) Appl. No.: 09/784,665

(22) Filed: Feb. 15, 2001

(51) Int. Cl. A61M 29/00

(52) U.S. Cl. 606/194

(57) ABSTRACT

A balloon catheter assembly including an inflation port having a seal disposed thereon to maintain the inflation lumen and balloon substantially free of air or other gas prior to use or during storage. Prior to use, the evacuated lumen is filled with liquid which releases the vacuum therein and brings the pressure up to at least atmospheric pressure without introducing air or other gases into the lumen, assuring a properly prepared balloon catheter free of air. A sealing device is provided which allows placing a seal on the port while pulling or maintaining vacuum on the lumen.

### FIELD OF THE INVENTION

The present invention generally relates to intravascular catheters, particularly balloon catheters. More precisely, the present invention relates to balloon catheters with a vacuum sealed inflation lumen.

### BACKGROUND OF THE INVENTION

The use of intravascular catheters has become an effective method for treating many types of vascular disease. In general, an intravascular catheter is inserted into the vascular system of the patient and navigated through the vasculature to a desired target site. Using this method, virtually any target site in the patient's vascular system may be accessed, including the coronary, cerebral, and peripheral vasculature. Examples of therapeutic purposes for intravascular catheters include percutaneous transluminal angioplasty (PTA) and percutaneous transluminal coronary angioplasty (PTCA).

Intravascular catheters are commonly used in conjunction with a guidewire. A guidewire may be advanced through the patient's vasculature until it has reached a target location. Once in place, a catheter may be threaded onto the guidewire and urged distally until the distal end of the catheter reaches a target location.

Intravascular catheters adapted for use with a guidewire typically are classified as over-the-wire (OTW) or single-operator-exchange (SOE). An OTW catheter includes a guidewire lumen extending from the distal tip of the catheter to the proximal end of the catheter. When intravascular catheters are used, it is common for physicians to remove one catheter and exchange it for another. While exchanging catheters, the guidewire should preferably be held in place so as to keep its distal end near the target area. A portion of the guidewire is typically grasped by the physician in order to withdraw the first catheter while maintaining the distal end of the guidewire in the desired position. To properly anchor the guidewire, a portion of the guidewire should preferably be exposed at all times so it is available for the physician to grasp. In the case of an OTW catheter, the length of the guidewire extending beyond the patient's body should be longer than the catheter. Consequently, in many cases, intravascular guidewires are longer than 200 cm or require guidewire extensions to facilitate exchange, and there may be more than 200 cm of wire extending from the patient. Managing this length of wire during a catheter exchange procedure can be awkward, and often requires more than one person.

SOE catheters overcome some of the difficulties encountered when exchanging OTW catheters. Accordingly, SOE catheters have a relatively short guidewire lumen relative to the length of the catheter. Therefore, the length of guidewire extending beyond the body of the patient need only be slightly longer than the guidewire lumen of the catheter. The physician may anchor or hold the guidewire as the first catheter is removed from the body, with the exchange occurring over the shorter guidewire lumen. The guidewire lumen of an SOE catheter typically includes a distal guidewire port disposed at the distal tip of the catheter and a proximal guidewire port disposed proximally of the distal end of the catheter.

When in use, intravascular catheters enter a patient's vasculature at a convenient location and then are urged to a target region. Once the distal portion of the catheter has entered the patient's vascular system, the physician may urge the distal tip forward by applying longitudinal forces to the proximal portion of the catheter. Then a physician may use the functional portion of the catheter to perform a medical procedure. For example, a physician may inflate an angioplasty balloon by passing a fluid through an inflation lumen.

Before a medical procedure can be performed, the catheter will need to be prepared. For example, an angioplasty balloon should be free of air before using an angioplasty catheter. The procedure for evacuating air from the balloon typically includes passing fluid through the inflation lumen into the balloon and then eliminating any air bubbles that may be present. To remove the air bubbles, a person might need to tap or bump the catheter to urge the bubbles out of the balloon. Not only can this procedure be time consuming, it can lead to kinks in a catheter shaft or cracked manifolds. For these reasons, a need exists for catheters that can reduce the amount of preparation time and reduce potential damage to the catheter during preparation.

Further, if the procedure is not conducted properly, there is no real assurance that the balloon is completely evacuated of air. This is an obvious problem if a balloon were to rupture in the vasculature. Further, this can be an important issue when precise balloon inflation is required. For example, air bubbles in an angioplasty balloon may alter the size or shape of the balloon once it is inflated. If the balloon is to be used in a sensitive tissue area, e.g., the central nervous system, alterations of the balloon can have major consequences. A need therefore exists for a catheter with increased assurance that the balloon is evacuated of air.

### SUMMARY OF THE INVENTION

The present invention generally relates to intravascular catheters, particularly balloon catheters. More precisely, the present invention relates to balloon catheters with a vacuum sealed inflation lumen. According to a preferred embodiment, the present invention comprises a catheter with substantially reduced preparation time and reduced potential damage to the catheter during preparation. Preferably, the catheter further comprises increased assurance that the balloon is evacuated of air or other gas.

According to a preferred embodiment, the present invention comprises a seal for the proximal entry port of an inflation lumen on a balloon catheter such as an over-the-wire catheter. In a preferred embodiment, an over-the-wire (OTW) catheter comprises an elongate member having a proximal end and a distal end. Preferably, a balloon is attached proximate the distal end.

In a preferred embodiment, the catheter further comprises a first tube including a proximal end, a distal end, and a first lumen extending therethrough. Preferably, the first tube defines the outside surface of the elongate member, or alternatively, the first tube can be disposed within the elongate member. In an exemplary embodiment, the first lumen is an inflation lumen for a balloon, such as an angioplasty balloon. According to a preferred embodiment, the first lumen is in fluid communication with a balloon.

In a preferred embodiment, the over-the-wire catheter further comprises a second tube including a proximal end, a distal end, and a second lumen extending therethrough. According to a preferred embodiment, the second tube is disposed within the elongate member or within the first tube. Preferably, the second lumen is a guidewire lumen adapted for receiving a guidewire.

In a preferred embodiment, the over-the-wire catheter further comprises a first port and a second port. Preferably, the first port is disposed at the proximal end of the first tube and is in fluid communication with the first lumen. Preferably, the second port is disposed at the proximal end of the second tube and is in fluid communication with the second lumen. In a preferred embodiment, a seal is operatively affixed to the first port. Preferably, the seal prevents fluids and air from entering the first lumen. In an exemplary embodiment, the seal prevents fluids and air from entering a balloon, for example an angioplasty balloon.

An alternative embodiment of the present invention comprises a vacuum seal for the proximal entry port of a balloon inflation lumen of a single-operator-exchange (SOE) catheter. According to a preferred embodiment, a single-operator-exchange catheter comprises an elongate member having a proximal end and a distal end. Preferably, a balloon is attached proximate the distal end.

In a preferred embodiment, the single-operator-exchange catheter further comprises a first tube including a proximal end, a distal end, and a first lumen extending therethrough. Preferably, the first tube defines the outside surface of the elongate member, or alternatively, the first tube can be disposed within the elongate member. In an exemplary embodiment, the first lumen is an inflation lumen for a balloon, such as an angioplasty balloon. In an exemplary embodiment, the first lumen is in fluid communication with a balloon.

In a preferred embodiment, the single-operator-exchange catheter further comprises a second tube including a proximal end, a distal end, and a second lumen extending therethrough. According to a preferred embodiment, the second tube is disposed within the first tube or within the elongate member. Preferably, the second lumen is a guidewire lumen adapted for receiving a guidewire.

In a preferred embodiment, the single-operator-exchange catheter further comprises a first port and a second port. Preferably, the first port is disposed at the proximal end of the first tube and is in fluid communication with the first lumen. In a preferred embodiment, a seal is attached to the first port. Preferably, the seal prevents fluids and air from entering the first lumen. In an exemplary embodiment, the seal prevents fluids and air from entering a balloon, such as an angioplasty balloon.

According to a preferred embodiment, the single-operator-exchange catheter further comprises a second port disposed at the proximal end of the second tube. Preferably, the second port is a guidewire port and is near the distal end of the single-operator-exchange catheter proximal to the balloon. Preferably, the second port in fluid communication with the second lumen.

An exemplary embodiment of the present invention includes a preferred method of attaching a vacuum seal to a catheter. The first port is adapted to receive an attachment, such as a sealing device. In a preferred embodiment, a sealing device is adapted for sealing a first port of an over-the-wire or single-operator-exchange catheter. Preferably, the first port further comprises a first lumen and at least one flange. Preferably, the sealing device further comprises a distal end that engages a flange of the first port. Preferably, engagement of the distal end and the flange constitutes an air-tight seal.

In a preferred embodiment, the sealing device further includes a vacuum lumen therethrough. Preferably, the vacuum lumen connects a chamber within the sealing device to a proximal end of the sealing device. Preferably, the proximal end is adapted for attaching to a vacuum source.

In a preferred embodiment, the chamber is defined as the space formed between the sealing device and the first port when the sealing device is attached to the first port. Preferably, disposed within the chamber is a seal attachment means attached to a mid-region of the sealing device. Preferably, a seal is releasably attached to the seal attachment means. The seal attachment means is movable from a first position spaced apart from the flange to allow flow of air out of the inflation lumen to a second position wherein the seal engages the flange and seals the inflation lumen.

In a preferred embodiment, the vacuum source may pull a vacuum through the vacuum lumen and inflation lumen when the sealing device is disposed on the flange. Application of a vacuum draws air out of the inflation lumen and balloon. The seal attachment means includes threads or other means for advancing the seal to engage the flange via the seal attachment means moving from the first position to the second position. The seal can include adhesive which bonds the seal to the flange when placed in contact. Preferably, the air pressure in the first lumen prior to the transfer of the seal is about zero. In an exemplary embodiment, after the transfer of the seal to the first port, the air pressure within the first lumen is about zero.

Preferably, the seal, as positioned on the flange, is adapted for receiving additional preparation devices. For example, an additional preparation device may include a syringe for placing a substance into the first lumen. Preferably, the syringe comprises a needle capable of piercing the seal. In an exemplary embodiment, the seal comprises a self-resealing septum. In a preferred embodiment, after piercing the seal, the syringe can deliver a fluid for inflation through the needle into the first lumen. This relieves the vacuum from the lumen by displacing the voided volume with fluid, not air or other gas, and easily completes the preparation for use with certainty that gas is not present in the inflation lumen or balloon. Once the vacuum is relieved with fluid, the seal can be removed.

In an alternative embodiment, the seal can be a self-resealing septum. In this embodiment, the seal is placed on the flange, followed by pulling vacuum through a needle which pierces the septum. When the inflation lumen and balloon are evacuated, the needle is pulled out of the self-resealing septum which maintains the vacuum.

An exemplary embodiment of the present invention includes an alternative seal for use with over-the-wire and single-operator-exchange catheters. Preferably, a seal is attached to the first port and is capable of maintaining an air-tight seal with the first lumen. According to this embodiment, the seal includes a cap which can be releasably attached to the first port and over the seal.

An alternative embodiment of the invention includes an alternative seal for use with over-the-wire and single-operator-exchange catheters. Preferably, a seal is attached to the first port and is capable of maintaining an air-tight seal with the first lumen. According to this embodiment, the seal is generally oversized relative to the first port. Preferably, an excess portion of the seal extends past at least one border of the first port. Preferably, the excess portion may be grasped by the fingers of a person or by a suitable grasping device so that the seal can be removed from the first port.

An alternative embodiment of the invention includes an alternative seal for use with over-the-wire and single-operator-exchange catheters. Preferably, a seal is attached to the first port and is capable of maintaining an air-tight seal with the first lumen. According to this embodiment, the seal further comprises threads capable of releasably attaching the seal to the first port.

An alternative embodiment of the invention includes an alternative seal for use with over-the-wire and single-operator-exchange catheters. Preferably, a seal is attached to the first port and is capable of maintaining an air-tight seal with the first lumen. According to this embodiment, the seal further comprises a tapered distal end capable of releasably attaching to the first port.

### BRIEF DESCRIPTION OF THE DRAWINGS

FIG. 1 is a partial cross-sectional view of a catheter including a vacuum seal for use with over-the-wire catheters according to a preferred embodiment of the invention;

FIG. 2 is a partial cross-sectional view of a catheter including a vacuum seal for use with single-operator-exchange catheters according to a preferred embodiment of the invention;

FIG. 3 is an illustration of a preferred mechanism for attaching a vacuum seal to a catheter, with the seal attachment means in a first position holding the seal spaced from the flange;

FIG. 4 is an illustration of the mechanism for attaching a vacuum seal to a catheter of FIG. 3, with the seal attachment means in a second position holding the seal in sealing engagement with the flange;

FIG. 5 is a diagrammatic view of a first vacuum seal according to a preferred embodiment of the invention;

FIG. 6 is a diagrammatic plan view of a vacuum seal and a needle/syringe assembly according to a preferred embodiment of the invention;

FIG. 7 is an enlarged view of an alternative vacuum seal and cap assembly according to a preferred embodiment of the invention;

FIG. 8 is an enlarged view of an alternative vacuum seal according to a preferred embodiment of the invention;

FIG. 9 is an enlarged view of an alternative vacuum seal according to a preferred embodiment of the invention; and

FIG. 10 is an enlarged view of an alternative vacuum seal according to a preferred embodiment of the invention.

### DETAILED DESCRIPTION OF THE PREFERRED EMBODIMENTS

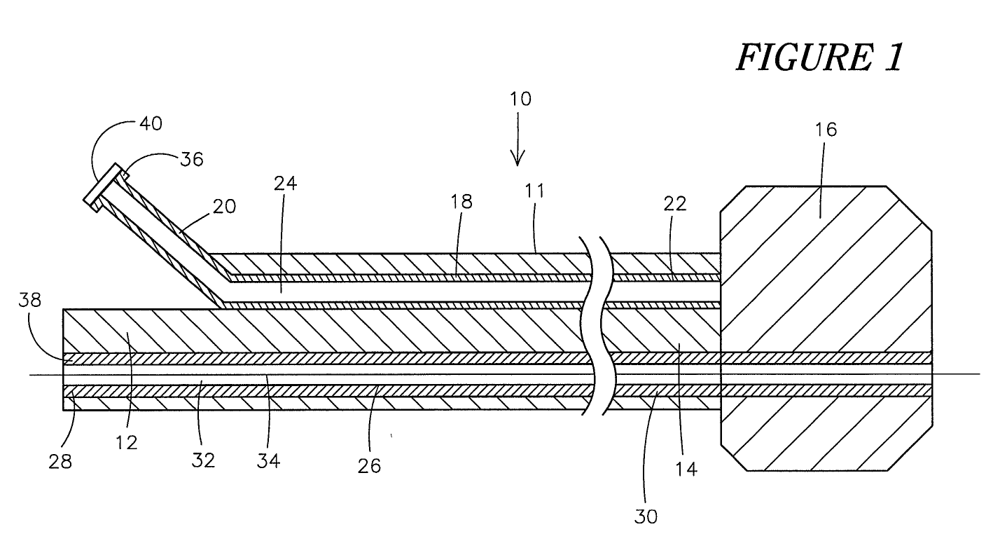

Referring now to the drawings wherein like reference numerals indicate like elements throughout the several views, FIG. 1 is a highly diagrammatic partial cross-sectional view of a catheter including a vacuum seal. The catheter is an over-the-wire catheter. According to a preferred embodiment, an over-the-wire catheter 10 comprises an elongate member 11 having a proximal end 12 and a distal end 14. Preferably, a balloon 16 is attached proximate the distal end 14. According to a preferred embodiment, elongate member 11 can be manufactured from materials including, but not limited to, metal, stainless steel, nickel alloys, nickel-titanium alloys, nitinol, hypodermic tubing, hollow cylindrical stock, polymers, plastics, and combinations thereof.

In a preferred embodiment, catheter 10 further comprises a first tube 18 including a proximal end 20, a distal end 22, and a first lumen 24 extending therethrough. According to a preferred embodiment, first tube 18 is disposed within elongate member 11. Preferably, first lumen 24 is an inflation lumen. In an alternative embodiment, the first tube and the elongate member may be a single tubular member. According to both embodiments, first lumen 24 is in fluid communication with balloon 16. In use, first lumen 24 is filled with an inflation fluid to inflate the balloon 16. Preferably, first tube 18 is manufactured from materials including, but not limited to, metal, stainless steel, nickel alloys, nickel-titanium alloys, nitinol, hypodermic tubing, hollow cylindrical stock, polymers, plastics, and combinations thereof.

According to an exemplary embodiment, first tube 18 may include a chemical coating capable of binding air including carbon dioxide (CO₂), nitrogen (N₂), and oxygen (O₂). Preferably, the chemical coating may help maintain the air pressure within first tube 18 at about zero. The chemical coating may include substances sold commercially which are called “getters”. A person of ordinary skill in the art would be familiar with a getter and the use thereof according to multiple embodiments of the present invention.

In a preferred embodiment, catheter 10 further comprises a second tube 26 including a proximal end 28, a distal end 30, and a second lumen 32 extending therethrough. Preferably, second tube 26 is disposed within elongate member 11, or alternatively, disposed within the first tube. According to a preferred embodiment, second lumen 32 is a guidewire lumen adapted for receiving a guidewire 34. Preferably, second tube 26 can be manufactured from materials including, but not limited to, metal, stainless steel, nickel alloys, nickel-titanium alloys, nitinol, hypodermic tubing, hollow cylindrical stock, polymers, plastics, and combinations thereof.

In a preferred embodiment, catheter 10 further comprises a first port 36 and a second port 38. Preferably, first port 36 is disposed at proximal end 20 of first tube 18 and is in fluid communication with first lumen 24. Preferably, second port 38 is disposed at proximal end 28 of second tube 26 and is in fluid communication with second lumen 32.

In a preferred embodiment, a seal 40 is attached to first port 36, covering the opening thereto. Preferably, seal 40 prevents fluids and air from entering and/or exiting first lumen 24. According to a preferred embodiment, seal 40 may comprise a polymer, rubber, a rubber septum, or plastic. Polymers include, but are not limited to, thermoplastics, high performance engineering resins, polyethylene (PE), polypropylene (PP), polyvinylchloride (PVC), polyurethane, polytetrafluoroethylene (PTFE), polyetherether ketone (PEEK), polyimide, polyamide, polyphenylene sulfide (PPS), polyphenylene oxide (PPO), polysufone, nylon, or perfluor(opropyl vinyl ether) (PFA).

In one preferred embodiment, the seal 40 is a rubber septum, wherein a penetrating member can pass through the seal, for example a needle, that upon removal of the penetrating member, the seal will self-seal. In an exemplary embodiment, a needle can be used to deliver a fluid into first lumen 24. Alternatively, a needle can be used to pull vacuum on the lumen when it has penetrated the seal. According to multiple embodiments of the present invention, self-seal is understood to mean that a seal, for example seal 40, will remain substantially resistant to the passage of air or fluids after a selected penetrating member is removed from the seal.

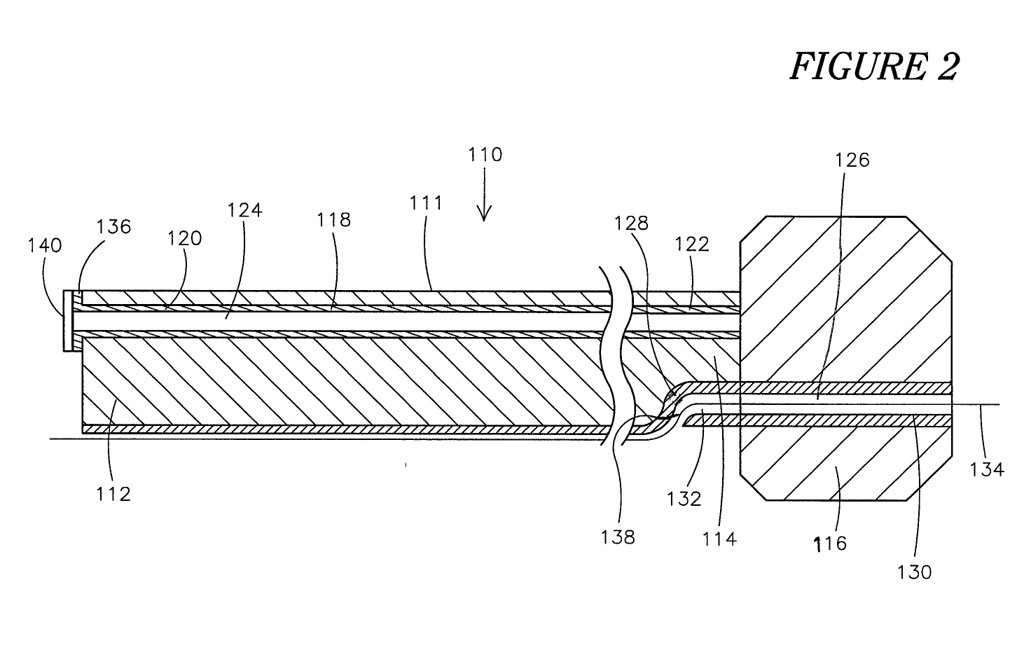

FIG. 2 is a highly diagrammatic partial cross-sectional view of a catheter having a vacuum seal. The catheter is a single-operator-exchange catheter. According to a preferred embodiment, a single-operator-exchange catheter 110 comprises an elongate member 111 having a proximal end 112 and a distal end 114. Preferably, a balloon 116 is attached proximate the distal end 118. According to a preferred embodiment, elongate member 111 is manufactured from materials including, but not limited to, metal, stainless steel, nickel alloys, nickel-titanium alloys, nitinol, hypodermic tubing, hollow cylindrical stock, polymers, plastics, and combinations thereof.

In a preferred embodiment, catheter 110 further comprises a first tube 118 including a proximal end 120, a distal end 122, and a first lumen 124 extending therethrough. According to a preferred embodiment, first tube 118 is disposed within elongate member 111. Alternatively, the elongate member and the first tube can be a single tubular member having a lumen therethrough. Preferably, first lumen 124 is an inflation lumen. According to this embodiment, first lumen 124 is in fluid communication with balloon 116. In an exemplary embodiment, in use, first lumen 124 is filled with a fluid, for example an inflation fluid. Similar to what is disclosed above, first tube 118 is preferably manufactured from materials including, but not limited to, metal, stainless steel, nickel alloys, nickel-titanium alloys, nitinol, hypodermic tubing, hollow cylindrical stock, polymers, plastics, and combinations thereof.

In a preferred embodiment, catheter 110 further comprises a second tube 126 including a proximal end 128, a distal end 130, and a second lumen 132 extending therethrough. Second tube 126 is disposed within elongate member 111 or within the first tube of a single tubular member. Preferably, second lumen 132 is a guidewire lumen adapted for receiving a guidewire 134. In the depicted single-operator-exchange embodiment, the second tube 126 extends over only a portion of the length of the catheter. The proximal end of the second tube is located a short distance proximal of the balloon. Similar to what is disclosed above, second tube 126 is preferably manufactured from materials including, but not limited to, metal, stainless steel, nickel alloys, nickel-titanium alloys, nitinol, hypodermic tubing, hollow cylindrical stock, polymers, plastics, and combinations thereof.

In a preferred embodiment, catheter 110 further comprises a first port 136 and a second port 138. Preferably, first port 136 is disposed at proximal end 120 of first tube 118 and is in fluid communication with first lumen 124. Preferably, second port 138 is disposed at proximal end 128 of second tube 126 and is in fluid communication with second lumen 132. Preferably, second port 138 is guidewire port.

In a preferred embodiment, a seal 140 is attached to first port 136. Preferably, seal 140 prevents fluids and air from entering and/or exiting first lumen 124. Similar to what is disclosed above, seal 140 preferably comprises a polymer, rubber, a rubber septum, or plastic.

First tube 118 may include a chemical coating capable of binding air including carbon dioxide (CO₂), nitrogen (N₂), and oxygen (O₂). Preferably, the chemical coating may help maintain the vacuum within first tube 118 at an air pressure about zero. The chemical coating may include substances sold commercially called getters. A person of ordinary skill in the art would be familiar with a getter and the use thereof according to multiple embodiments of the present invention.

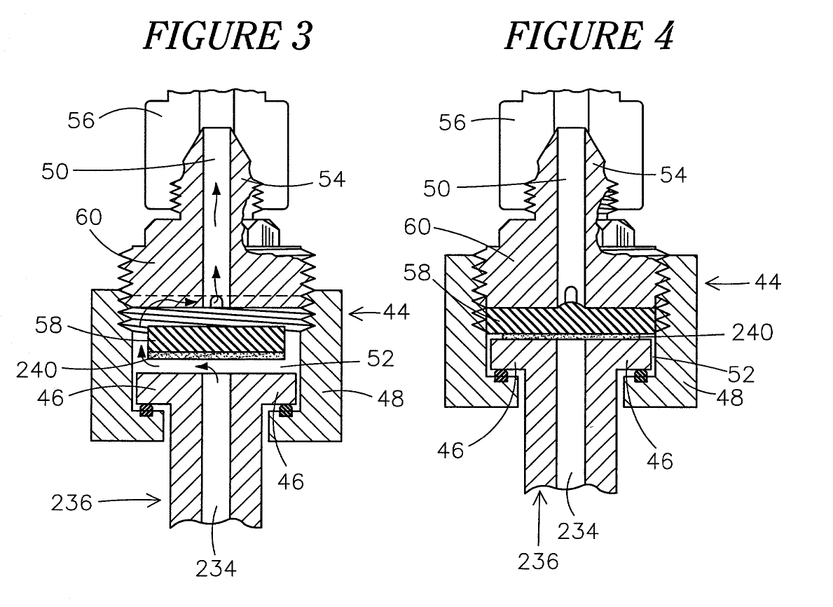

FIG. 3 is an illustration of a preferred apparatus to be utilized in a method of attaching a vacuum seal 58 to a catheter port 236. In a preferred embodiment, an attachment 44, for example a sealing device, is adapted for sealing first port 236 comprising first lumen 234 and at least one flange 46. Preferably, attachment 44 further comprises a distal end 48 that engages flange 46 of first port 236. Preferably, engagement of distal end 48 and flange 46 constitutes an air-tight seal.

In a preferred embodiment, attachment 44 further comprises a vacuum lumen 50. Preferably, vacuum lumen 50 connects a chamber 52 to a proximal end 54 of attachment 44. Preferably, proximal end 54 is adapted for attaching to a vacuum source 56.

In a preferred embodiment, chamber 52 is defined as the space formed between attachment 44 and first port 236 when attachment 44 is attached to first port 236. Preferably, disposed within chamber 52 is a seal attachment means 58 attached to a movable member 60 of attachment 44. Preferably, a seal 240 is releasably attached to seal attachment means 58. Similar to what is disclosed above, seal 240 preferably comprises a polymer, rubber, a rubber septum, and plastic.

As indicated in FIG. 3, a seal attachment means 58 is mounted on the end of a movable member 60. The movable member 60 is depicted in FIG. 3 as being retracted away from the flange 46 so that when vacuum is pulled on the lumen 50, air or other gas flows out of the inflation lumen of the catheter via the first port. In this first position of the movable member 60, the catheter can be put under sufficient vacuum to remove most, if not substantially all, of the air or other gas from the inflation lumen and balloon of the catheter.

FIG. 4 is an illustration of the preferred apparatus for attaching a vacuum seal to a catheter of FIG. 3 showing the way in which the seal 240 is placed on the flange 46. With the apparatus as configured in FIG. 3, one may pull a vacuum through vacuum lumen 50. Application of a vacuum forces air or gas out of the inflation lumen and balloon. Once sufficient vacuum has been pulled, movable member 60 is advanced, via threads, to the second position indicated in FIG. 4 to transfer seal 240 to first port 236. Although threads are used to advance moveable member 60, it is recognized that other means known by those in the art can be utilized. Preferably, the air pressure in first lumen 234 just prior to the transfer of seal 240 is about zero. In an exemplary embodiment, after the transfer of seal 240 to first port 236, the air pressure within first lumen 234 is about zero.

As discussed above, attachment 44 includes a device adapted to attach to the first port and that can evacuate the first lumen and attach a seal so that vacuum is retained.

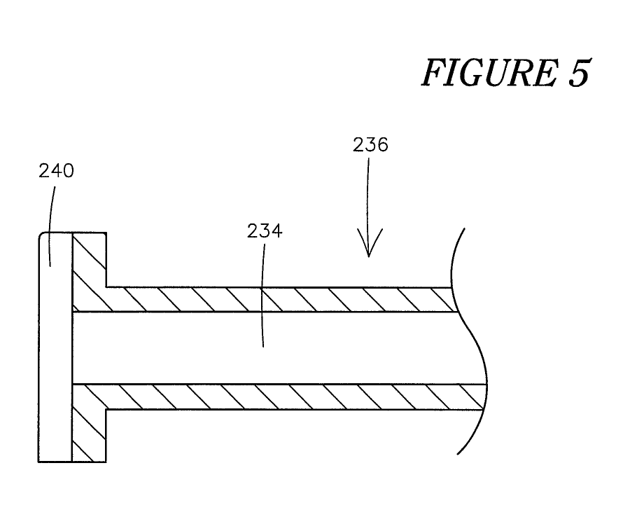

FIG. 5 depicts the sealed port upon removal of the attachment 44. According to a preferred embodiment, first port 236 comprises first lumen 234. Seal 240 is attached to first port 236 and is capable of maintaining an airtight seal within first lumen 234.

In preferred embodiments, the catheter having an evacuated and sealed inflation lumen is shipped to the end-user. However, before use, all that must be done is to fill the evacuated lumen with inflation fluid as vacuum is relieved. In a preferred embodiment, a device penetrates the seal to release an inflation fluid into the first lumen. In alternate embodiments, a device may be adapted to remove a seal and simultaneously connect to an inflation device and its fluid source. In an exemplary embodiment, a device may comprise an object that can pierce the seal, for example a needle. Alternatively, the attachment may comprise an object capable of breaking the seal. For example, the device may include a ramming portion that forcibly breaks the seal. In alternative embodiments of the current invention, a device may accomplish any combination of the features listed above.

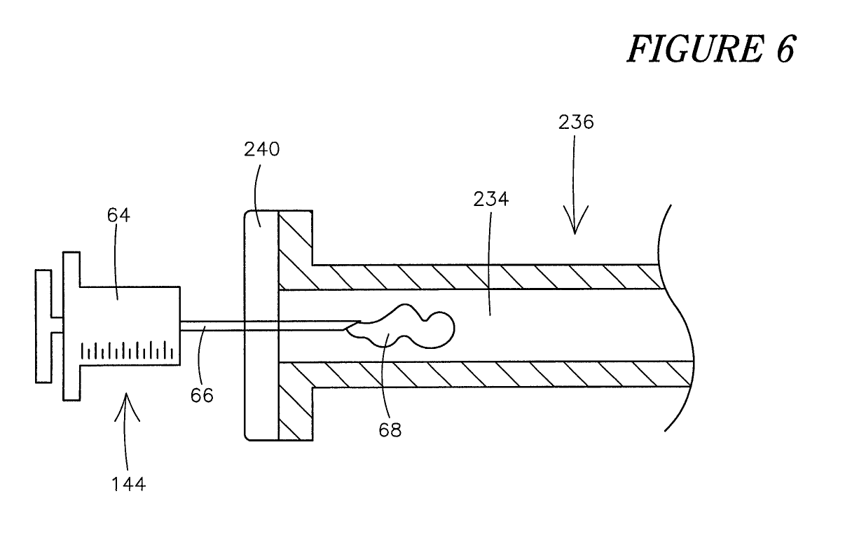

FIG. 6 is a diagrammatic view of a preferred apparatus and method of penetrating a vacuum seal to fill the lumen with inflation fluid according to a preferred embodiment of the invention. According to preferred embodiment, first port 236 comprises first lumen 234. Preferably, seal 240 is attached to first port 236 and is capable of maintaining an airtight seal within first lumen 234. Preferably, first port 236 is adapted for receiving a seal piercing device 144. For example, device 144 may comprise a syringe 64 for placing a substance such as an inflation fluid into first lumen 236 to relieve the vacuum and fill the lumen and balloon with inflation fluid 68. Preferably, syringe 64 comprises a needle 66 capable of piercing seal 240. In a preferred embodiment, after piercing seal 240, syringe 64 can deliver a fluid 68 through needle 66 into first lumen 234. After the lumen is filled, seal 240 can be removed to facilitate attachment of another inflation device. Alternatively, the inflation device can incorporate means for piercing the seal when in use.

The embodiment depicted in FIG. 6 also illustrates an alternative method for evacuating the inflation lumen of a catheter without utilizing the above-described seal placement device. In particular, the catheter port 236, including a lumen 234 therein which is in fluid communication with the balloon, can have a seal 240 placed thereon while the lumen is still full of air or other gas. In this embodiment, the seal is made of a material that can be penetrated, but reseals upon removal of the penetrating device. Thus, the seal 240 is adhered to the flange of the port 236 and the penetrating member or needle 66 pierces the seal. A source of vacuum, such as the syringe 64 or other vacuum producing mechanism, is attached to the proximal end of the needle. Vacuum may then be drawn so that air or other gas is evacuated from the inflation lumen. When sufficient vacuum has been pulled, the needle 66 may be pulled back out of the seal 240 which then reseals to hold the lumen 234 as evacuated.

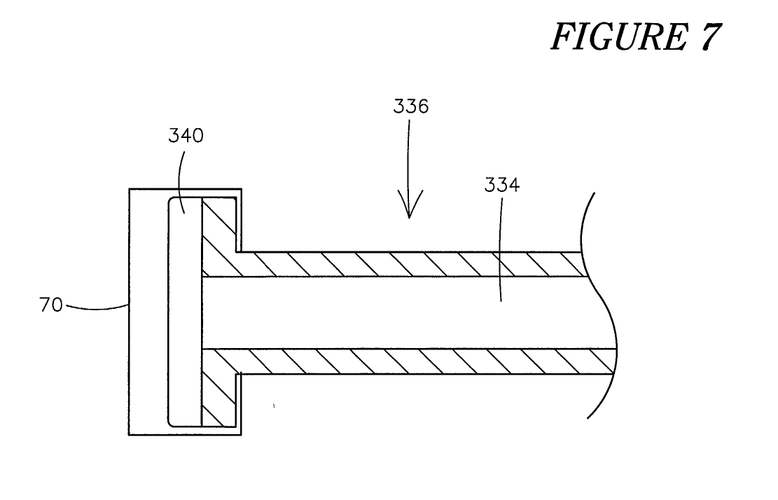

FIG. 7 is an enlarged view of an alternative embodiment of a vacuum seal. According to this embodiment, first port 336 comprises first lumen 334. Preferably, seal 340 is attached to first port 336 and is capable of maintaining a relatively air-tight seal within first lumen 334. According to this embodiment, a cap 70 is releasably attached to first port 336 and over seal 340. Similar to what is disclosed above, seal 340 preferably comprises a polymer, rubber, a rubber septum, and plastic. The cap assembly is incorporated to provide additional assurance that the lumen 334 will remain evacuated using any of the above methods of evacuation, even when the evacuated product is stored for an extended period. The use of the cap is especially useful with methods that include piercing the seal to pull vacuum, as it is believed that a perfect seal may not be maintained in some instances when the seal re-seals.

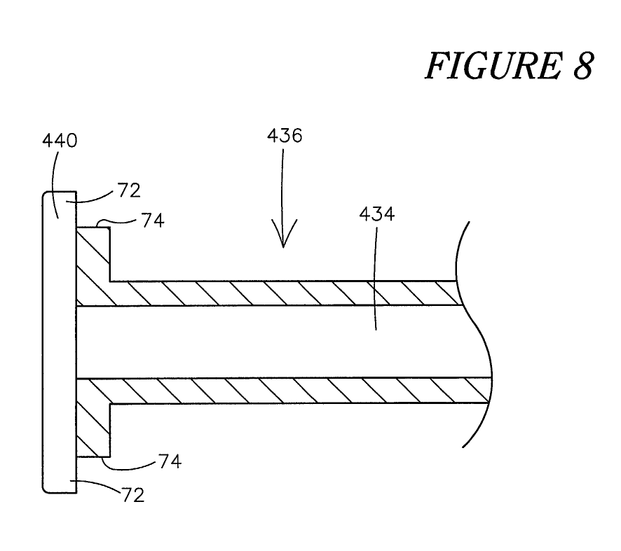

FIG. 8 is a diagrammatic view of an alternative embodiment of a vacuum seal. According to this embodiment, first port 436 comprises first lumen 434. Preferably, seal 440 is attached to first port 436 and is capable of maintaining an air-tight seal within first lumen 434. Similar to what is disclosed above, seal 440 preferably comprises a polymer, rubber, a rubber septum, and plastic. According to this embodiment, seal 440 is generally oversized relative to first port 436. Preferably, an excess portion 72 of seal 440 extends past at least one border 74 of first port 436. Preferably, excess portion 72 may be grasped by the fingers of a person or by a suitable grasping device so that seal 440 can be removed from first port 436. This facilitates seal removal after inflation fluid has been added to the evacuated lumen.

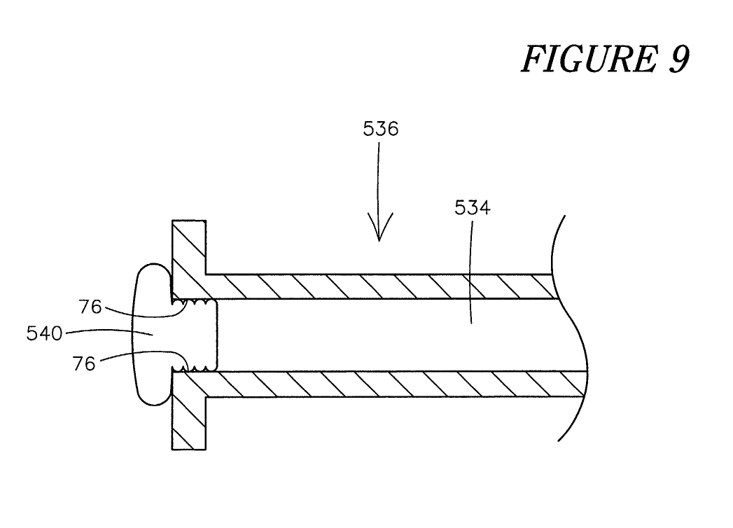

FIG. 9 is a diagrammatic view of an alternative embodiment of a vacuum seal. According to this embodiment, first port 536 comprises first lumen 534. Preferably, seal 540 is attached to first port 536 and is capable of maintaining an air-tight seal within first lumen 534. Similar to what is disclosed above, seal 540 preferably comprises a polymer, rubber, a rubber septum, and plastic. According to this embodiment, seal 540 further comprises threads or ridges 76 capable of releasably attaching seal 540 to first port 536.

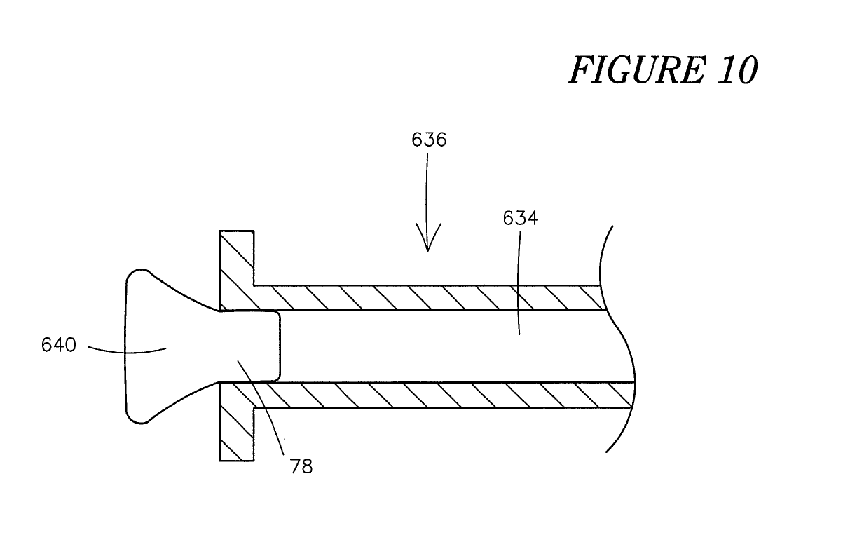

FIG. 10 is a diagrammatic view of an alternate embodiment of a vacuum seal. According to this embodiment, first port 636 comprises first lumen 634. Preferably, seal 640 is attached to first port 636 and is capable of maintaining an air-tight seal within first lumen 634. Similar to what is disclosed above, seal 640 preferably comprises a polymer, rubber, a rubber septum, and plastic. According to this embodiment, seal 640 further comprises a tapered distal end 78 capable of releasably attaching to first port 636.

Numerous advantages of the invention covered by this document have been set forth in the foregoing description. It will be understood, however, that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A catheter assembly, comprising:
a first tube having a proximal end, a distal end, and a first lumen extending therethrough, wherein the lumen is in fluid communication with an expandable member proximate the distal end of the first tube;
a first port on the proximal end of the first tube in fluid communication with the first lumen; and
a seal releasably attached to the first port so as to substantially prevent the passage of air into the first lumen, wherein the first lumen has a pressure less than atmospheric pressure.

2. The catheter assembly in accordance with claim 1, wherein the expandable member is a balloon.

3. The catheter assembly in accordance with claim 1, wherein the seal comprises a polymer.

4. The catheter assembly in accordance with claim 1, wherein the seal comprises rubber.

5. The catheter assembly in accordance with claim 4, wherein the seal comprises a self-sealing rubber septum.

6. The catheter assembly in accordance with claim 1, wherein the seal comprises plastic.

7. The catheter assembly in accordance with claim 1, wherein the first tube includes a chemical coating capable of binding a quantity of CO₂, N₂, and O₂.

8. The catheter assembly in accordance with claim 1, wherein prior to use the first lumen is filled with a fluid.

9. A balloon catheter with a proximal end and a distal end, comprising:
a first tube having a proximal end, a distal end, and a first lumen extending therethrough, wherein the first tube includes a first port on a proximal end thereof in fluid communication with the first lumen;
a balloon disposed at the distal end of the catheter and in fluid communication with the first lumen; and
a seal releasably attached to the first port so as to substantially prevent the passage of air into the first lumen wherein the first lumen is substantially free of air therein.

10. The catheter in accordance with claim 9, wherein the seal comprises a polymer.

11. The catheter in accordance with claim 9, wherein the seal comprises rubber.

12. The catheter in accordance with claim 9, wherein the seal comprises a self-sealing rubber septum.

13. The catheter in accordance with claim 9, wherein the seal comprises plastic.

14. The catheter in accordance with claim 9, wherein the first tube includes a chemical coating capable of binding a quantity of CO₂, N₂, and O₂.

15. The catheter in accordance with claim 9, wherein prior to use the first lumen is filled with a fluid.

16. A method of preparing a balloon catheter, comprising the steps of:
providing a balloon catheter including a first tube having a proximal end, a distal end, and a first lumen extending therethrough, wherein the first tube has a first port in fluid communication with the first lumen, and a seal releasably attached to the first port so as to substantially prevent the passage of air into the first lumen with a balloon disposed proximate the distal end of the catheter in fluid communication with the first lumen;
providing a sealing device that is detachably connectable to the first port, wherein the sealing device includes a seal detachably secured therein;
connecting the sealing device to the first port;
using the sealing device to pull vacuum until the air pressure within the first lumen is substantially less than atmospheric pressure followed by placing the seal over the first port; and
disconnecting the sealing device from the first port.

17. The method in accordance with claim 16, wherein the seal comprises a polymer.

18. The method in accordance with claim 16, wherein the seal comprises rubber.

19. The method in accordance with claim 16, wherein the seal comprises a self-sealing rubber septum.

20. The method in accordance with claim 16, wherein the seal comprises plastic.

21. The method in accordance with claim 16, wherein the first tube includes a chemical coating capable of binding a quantity Of CO₂, N₂, and O₂.
